(19) United States
(12) Patent Application Publication
(10) Pub. No.: US 2013/0131470 A1
(43) Pub. Date: May 23, 2013

(54) METHODS AND SYSTEMS FOR IN VIVO CLINICAL DATA MEASUREMENT OF ANALYTES

(71) Applicants: Jeffrey Galinkin, Greenwood Village, CO (US); Uwe Christians, Evergreen, CO (US); Keith Hoffman, Aurora, CO (US)

(72) Inventors: Jeffrey Galinkin, Greenwood Village, CO (US); Uwe Christians, Evergreen, CO (US); Keith Hoffman, Aurora, CO (US)

(73) Assignee: The Regents of the University of Colorado, a body coporate, Denver, CO (US)

(21) Appl. No.: 13/809,892

(22) PCT Filed: Jul. 14, 2011
(86) PCT No.: PCT/US11/44039

§ 371 Date: Jan. 23, 2013

Related U.S. Application Data
(60) Provisional application No. 61/364,103, filed on Jul. 14, 2010.

(51) Int. Cl. A61B 5/145

(52) U.S. Cl. CPC A61B 5/14528
USPC 600/310

(57) ABSTRACT

The present invention provides apparatuses, systems, and methods for real-time measuring of analytes in a biological fluid sample of a subject. In particular, the present invention provides a combination of micro-dialysis catheter, a micro-volume pump, and a spectrometer device that are operatively connected to one another to provide real-time measurement of analytes in a biological fluid sample of a subject.

### CROSS-REFERENCE TO RELATED APPLICATIONS

This application claims the priority benefit of U.S. Provisional Application No. 61/364,103, filed Jul. 14, 2010, which is incorporated herein by reference in its entirety.

### FIELD OF THE INVENTION

The present invention relates to apparatuses, systems, and methods for real-time measuring of analytes in a biological fluid sample of a subject. In particular, the present invention relates to using a combination of micro-dialysis catheter, a micro-volume pump, and a spectrometer device that are operatively connected to one another to provide real-time measurement of analytes in a biological fluid sample of a subject.

### BACKGROUND OF THE INVENTION

Clinical in vivo drug data from a subject provides a variety of information related to a drug that is administered to a subject. The terms “clinical in vivo drug data,” “in vivo clinical drug data” and “clinical drug data” are used interchangeably herein and include pharmacokinetics (PKs), data associated with clinical therapeutic drug monitoring, data associated with toxicology monitoring of a drug such as endogenous compounds that serve as surrogate markers of drug effect/toxicity (biomarkers), as well as any other useful information in the development, therapeutic, and/or use of the drug or in general any information that can be obtained or deduced from a subject by analyzing a fluid sample from the subject after administration of a drug. As used herein, the term “subject” or “patient” refers to any organism whose fluid is to be analyzed, e.g., for obtaining pharmacokinetics data, clinical therapeutic drug monitoring and toxicology monitoring, etc. Typical subjects include animals such as mammals including, but not limited to, mice, rats, rabbits, pigs, equines, bovines, dogs, cats, non-human primates, and humans.

Pharmacokinetics refers to what a subject's body does to a drug, that is, how the body processes the drug. Clinical therapeutic drug monitoring and/or drug toxicology monitoring (pharmacokinetic, pharmacodynamic, toxicokinetic, and/or toxicodynamic monitoring) are often used to detect levels of drugs in point of care and experimental testing as well as to determine toxicity and side-effects of the drug. Pharmacokinetic data may provide information related to the mechanism of drug absorption and distribution, drug metabolism, drug half-life, chemical changes of substances in the body, and effects and routes of excretion of drug metabolites. Pharmacokinetic data are often used in drug design, administration, determining proper dosing levels of pharmaceuticals, and for gathering efficacy and toxicology data. For example, the data is useful in pre-clinical animal studies and during Phase I and II clinical trials. PK data may also be useful in preventing or reducing cytotoxic effects during chemotherapy treatment.

Sampling (sample collection) for pharmacokinetics, clinical therapeutic analysis, or toxicology analysis is generally performed by either placing an indwelling intravenous or arterial line into a patient or more commonly, by performing multiple blood draws over time. The time points of the blood draws are usually tightly clustered around the time of drug administration and then become less frequent.

These blood samples then typically undergo quantitative or qualitative analysis by high-performance liquid chromatography (HPLC) or mass-spectrometry or other spectrometer based detection system. The levels of analyte detected at various time periods are then analyzed, typically by a graphing a curve of the results. These curves provide the pharmacokinetics information, clinical therapeutic information, or toxicology information of the drug. Sampling of a fluid can also be used to determine in vivo level of drug as well as rate of drug absorption to aid in a proper amount of drug to be administered in various treatments of clinical conditions such as, but not limited to, cancer, anti-coagulation (e.g., for administration of Warfrin), etc.

However, the accuracy of the curve and the usefulness of the information may be influenced by the accuracy of the sampling. For example, the frequency of sampling may be limited by, for example, the age of a patient or the current blood sampling protocols. That is, infants and children may have limited blood draws due to difficulty in accessing blood samples and limits on frequency and volume of blood draws.

Therefore, there is a need for a better device and/or method for sampling blood for pharmacokinetic analysis, clinical therapeutic drug monitoring and toxicology monitoring as well as other analytical uses.

### SUMMARY OF THE INVENTION

Some aspects of the invention provide apparatuses and methods for continuously sampling an analyte in a biological fluid. In some embodiments, methods of the invention include passing a fluid through a micro-dialysis catheter and measuring the level of analyte in real-time. In other embodiments, the micro-dialysis catheter is placed in an arterial or venous blood vessel. At least a portion of the micro-dialysis catheter is in fluid contact with the biological fluid such that the analyte in the biological fluid can be diffused into, e.g., the lumen of, the micro-dialysis catheter. By injection, perfusing, or infusing a dialysis buffer solution through the micro-dialysis using a pump, for example, a nanoflow or microflow pump, one can continuously monitor the level of analyte in real-time. As used herein, the term “continuously” refers to analytic measurements taken at a frequency of about once an hour or less, typically once every 30 minutes or less, often once every 10 minutes or less, and more often once a minute or less. The outlet of the micro-dialysis catheter can be connected to a second pump, e.g., an infusion pump, such that the dialysate is transported from the outlet of the micro-dialysis catheter to an analytical device to analyze the dialysate. In some embodiments, at least a portion of the dialysate is subjected to an ionization process by flowing through an ionization device. Suitable ionization devices are well known to one skilled in the art and includes, but are not limited to, flow infusion chip systems such as micro- and nano-flow infusion chip systems. In some embodiments, the real-time measurement of the analyte is achieved by using a spectrometry based analytical device such as a mass spectrometer, a UV/VIS spectrometer, an infrared spectrometer, chemical, electrochemical or biological sensors, a nuclear magnetic resonance spectrometer, or a combination thereof.

Other aspects of the invention provide apparatuses that are capable of continuous sampling and analyzing an analyte in a biological fluid sample. Such apparatuses include a micro-dialysis catheter comprising a first portion for contacting the biological fluid, an inlet port operatively connected to a pump, and an outlet port operatively connected to an analytical device. In some embodiments, apparatuses can also include a second pump that is operatively connected to the outlet port of the micro-dialysis catheter. The second pump can also be operatively connected to an ionization device such as a flow infusion chip system. The dialysate passing through the flow infusion chip system is typically analyzed using a mass spectrometer to permit real-time measurement of the analyte.

Still other aspects of the invention include a system that is capable of continuously sampling an analyte in a biological fluid sample. Such systems include a micro-dialysis catheter comprising an inlet port and an outlet port. Systems can also include a pump operatively connected to the inlet port of the micro-dialysis catheter. Systems can also include a second pump operatively connected to the outlet of the micro-dialysis catheter. Systems can also include an ionization device operatively connected to the second pump. Systems can also include a spectrometry based detection system.

In one particular aspect of the invention, an apparatus is provided that comprises a micro-volume pump capable of pumping a micro volume of fluid per minute; a micro-dialysis catheter having an inlet port and an outlet port, wherein the inlet port is operatively connected to the micro-volume pump such that a dialysis buffer solution can be injected or infused into the micro-dialysis catheter through the inlet port using the micro-volume pump to produce a dialysate; and a spectrometer detection device operatively connected to the outlet port of the micro-dialysis catheter for analyzing the dialysate.

In some embodiments, the apparatus further comprises an ionization device operatively connected to the outlet port of the micro-dialysis catheter for ionizing at least a portion of the dialysate prior to being analyzed by the spectrometer detection device.

Yet in other embodiments, the apparatus further comprises a second micro-volume pump operatively connected to the outlet port of the micro-dialysis catheter for transporting the dialysate to the spectrometer detection device.

Still in other embodiments, the spectrometer detection device comprises a mass spectrometer, a UV/VIS spectrometer, an infrared spectrometer, chemical, electrochemical or biological sensors, a nuclear magnetic resonance spectrometer, or a combination thereof.

Another particular aspects of the invention provide a method for real-time monitoring of an analyte in a fluid sample of a subject. Such a method typically comprises:
injecting or infusing a dialysis buffer fluid using a micro-volume pump through an inlet port of a micro-dialysis catheter that is placed within the subject to produce a dialysate fluid, wherein the micro-dialysis catheter is in fluid communication with the fluid sample of the subject to be analyzed, and wherein the micro-dialysis catheter comprises a barrier medium that allows selective diffusion of the analyte to be detected from the fluid sample of the subject into the lumen of the micro-dialysis catheter and the fluid contained therein; and
analyzing the dialysate fluid using a spectrometer detection device that is operatively connected to an outlet port of the micro-dialysis catheter thereby monitoring the analyte in the fluid sample of the subject.

In some embodiments, the analyte in the fluid sample is continuously monitored.

Yet in other embodiments, the analyte in the fluid sample is monitored at least once per hour, typically at least once per 30 minutes, often at least once per 15 minutes, and more often at least once per 10 minutes.

Still in other embodiments, the spectrometer detection device comprises a mass spectrometer, a UV/VIS spectrometer, an infrared spectrometer, a chemical, electrochemical or biological sensor, a nuclear magnetic resonance spectrometer, or a combination thereof. Within these embodiments, in some instances the spectrometer detection device comprises a mass spectrometer. Within such instances, in some cases method can further comprise flowing at least a portion of the dialysate fluid through an ionization device connected to the outlet port of the micro-dialysis catheter to ionize at least a portion of the dialysate prior to analyzing the dialysate using the mass spectrometer. The ionization device can comprise a flow infusion chip system.

In some embodiments, such methods further comprise transporting the dialysate from the outlet port of the micro-dialysis catheter to the spectrometer detection device using a second micro-volume pump that is operatively connected to the outlet port of the micro-dialysis catheter. In some instances, the second micro-volume pump can also reduce or avoid positive pressure in the micro-dialysis catheter. In other instances, the second micro-volume pump can be used to deliver a fluid or solvent that improves the detection of the analyte.

Yet in other embodiments, the micro-dialysis catheter is placed within a blood vessel of the subject. Within these embodiments, in some instances the micro-dialysis catheter is placed in an arterial or venous blood vessel.

Still in other embodiments, the micro-dialysis catheter is placed within the brain, other tissues or the spinal fluid of the subject.

In other embodiments, the micro-volume pump comprises a nanoflow pump or a microflow pump.

Still other aspects of the invention provide methods for obtaining a clinical in vivo drug data from a subject. Such methods typically include:
(a) administering to the subject a drug whose clinical in vivo drug data is to be obtained;
(b) injecting or infusing a dialysis buffer fluid using a micro-volume pump through an inlet port of a micro-dialysis catheter that is placed within the blood vessel of the subject to produce a dialysate fluid, wherein the micro-dialysis catheter comprises a barrier medium that allows selective diffusion of an analyte that is indicative of the drug pharmacokinetics, pharmacodynamics, toxicokinetics, toxicodynamics, or a combination thereof into the lumen of the micro-dialysis catheter and the dialysis fluid contained therein;
(c) analyzing the dialysate fluid using a spectrometer detection device that is operatively connected to an outlet port of the micro-dialysis catheter to determine the level of analyte within the subject's blood; and
(d) repeating steps (b) and (c) to obtain the clinical in vivo drug data from the subject.

In some embodiments, the spectrometer detection device comprises a mass spectrometer, a UV/VIS spectrometer, an infrared spectrometer, a chemical, electrochemical or biological sensor, or a nuclear magnetic resonance spectrometer. Within these embodiments, in some instances the spectrometer detection device comprises a mass spectrometer. In such instances, in some cases methods for obtaining clinical in vivo drug data include flowing at least a portion of the dialysate fluid through an ionization device connected to the outlet port of the micro-dialysis catheter to ionize at least a portion of the dialysate prior to analyzing the dialysate using the mass spectrometer. Ionization devices are well known to one skilled in the art, and any of the known ionization devices can be used in methods of the invention. In some cases, the ionization device comprises flow infusion chip systems.

In other embodiments, methods for obtaining clinical in vivo drug data can also include transporting the dialysate from the outlet port of the micro-dialysis catheter to the spectrometer detection device using a second micro-volume pump operatively connected to the outlet port of the micro-dialysis catheter.

Yet in other embodiments, the clinical in vivo drug data comprises pharmacokinetics data of the drug.

Still in other embodiments, the clinical in vivo drug data comprises data associated with the effectiveness of treatment of a clinical condition including but not limited to pharmacodynamic and/or toxicodynamic surrogate markers (biomarkers).

### BRIEF DESCRIPTION OF THE DRAWINGS

FIG. 1 is a schematic illustration showing one particular embodiment of an apparatus for real-time, continuous sampling and analysis of a clinical in vivo data according to the present disclosure.

FIG. 2 is a simulated graphic of PK curve that is obtained by an apparatus and/or a method as disclosed herein.

FIG. 3 is an in vitro MRM scan of acetaminophen using a micro-dialysis/LC-MS/MS system in Na₂EDTA human whole blood.

### DETAILED DESCRIPTION

Some aspects of the invention provide apparatuses and systems that can be used to collect real-time in vivo clinical drug data. Apparatuses disclosed herein provide can also be used to continuous sample an analyte in a biological sample or fluid, such as human blood, and allows real-time in vivo clinical drug data sampling and recording (analysis) of the sample without the need for drawing blood samples for each analysis. Apparatuses of the invention can also include in vivo extraction of the sample via a dialysis catheter that is placed in the subject's fluid (e.g., blood, spinal fluid, brain, etc.) thereby enabling direct analysis (e.g., quantification or qualitative analysis) via a spectrometer device such as infusion mass spectrometry.

Some aspects of the invention provide apparatuses that can analyze the full range of drug distribution and in vivo clinical drug data immediately after drug administration. Additionally, in certain aspects, apparatuses of the invention can measure the fraction of the drug that is not bound to proteins. The “free fraction” of the drug is generally considered the active fraction.

Apparatuses of the invention also allow for a rapid reliable analytic solution (RRAS) for control of intravenous drug infusions, which can be useful to medical fields, such as anesthesia, where intravenous anesthesia has become increasingly prevalent and anesthesiologists rely on technology to provide generally immediate feedback of drug effect and level. While such monitoring is currently available for anesthetic gases utilizing an in-line gas chromatography system, currently no commercial apparatuses or methods are available for non-gaseous anesthetics.

Some aspects of the invention eliminate the need for a repeated blood draws during phase I and phase II pharmacokinetic drug testing. Due to the full range of pharmacokinetic samples and the high sensitivity of the spectrometry device, this invention allows for smaller patient sample sizes and more accurate and “true” pharmacokinetic information since this technology allows high-frequency sampling and eliminates the loss of information that occurs between blood draws using conventional pharmacokinetic sampling techniques.

Other aspects of the invention provide methods for using apparatuses disclosed herein for continuously sampling an analyte in a biological fluid sample, such as blood, spinal fluid, etc. As used herein, the term “analyte” is a broad term and is used in its ordinary sense and includes, without limitation, any chemical species, the presence or concentration of which is sought in the biological fluid sample. Analyte(s) include xenobiotic compounds and/or endogenous compounds.

The present invention will be described with regard to the accompanying drawings which assist in illustrating various features of the invention. In this regard, the present invention generally relates to apparatuses and methods for obtaining and analyzing in vivo clinical data. That is, the invention relates to apparatuses and methods for obtaining real-time in vivo clinical drug data.

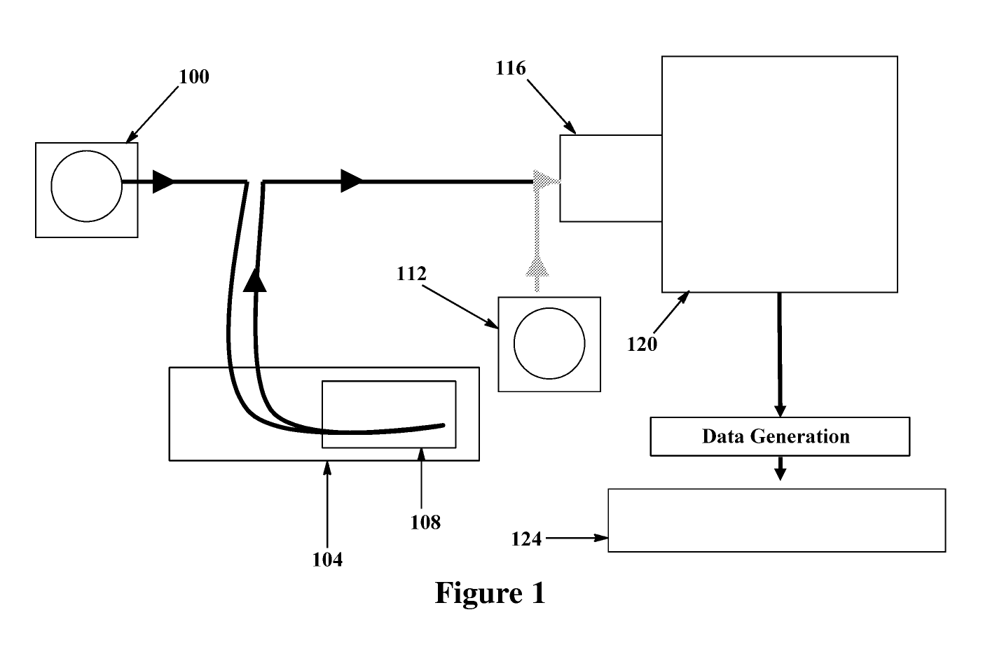

One particular embodiment of an apparatus for obtaining and analyzing in vivo clinical drug data is generally illustrated in FIG. 1, which is provided for the purpose of illustrating the practice of the present invention and does not constitute limitations on the scope thereof.

In FIG. 1, the apparatus of the invention includes a micro-volume pump 100 that is operatively connected to a micro-dialysis catheter 108 that is placed within a blood vessel 104 of a subject. Micro-volume pump 100 injects or infuses a dialysis buffer solution to micro-dialysis catheter 108. Micro-dialysis catheter 108 can also include a selective barrier (not shown) such as a semi-permeable membrane (not shown) that allows a selective diffusion of a desired analyte in the blood stream of the subject to diffuse into micro-dialysis catheter 108.

Micro-dialysis catheter 108 also includes an inlet port (not shown) that is operatively connected to micro-volume pump 100 such that the dialysis buffer solution pumped by micro-volume pump 100 is infused into micro-dialysis catheter 108. Micro-dialysis catheter 108 also includes an outlet port (not shown) which carries the dialysate comprising the analyte to be analyzed (if present) to a spectrometer device 120. Spectrometer device 120 can be any spectrometer based analytical device that can analyze the analyte. Exemplary spectrometer devices include, but are not limited to, a mass spectrometer, a chromatography device (such as high-performance liquid chromatography device, i.e., HPLC), an infrared spectrometer, a UV/VIS spectrometer, a chemical, electrochemical or biological sensor, a nuclear magnetic resonance spectrometer, or a combination thereof.

Referring again to FIG. 1, optionally apparatus can also include a second micro-volume pump 112 that is operatively connected to the outlet port of micro-dialysis catheter 108. In this manner, dialysate is transported to spectrometer device 120 at least in part by second micro-volume pump 112. Optionally, prior to analysis by spectrometer device 120, the dialysate can be subjected to a sample preparation using a sample preparation device 116, such as an ionization interface when spectrometer device 120 is a mass spectrometer. Data that is generated from spectrometer device 120 can optionally be analyzed automatically by a computer or any suitable data analysis device 124. Micro-dialysis catheter 108, micro-volume pump 100 and optional second micro-volume pump 112 are sometimes collectively referred to herein as a sampling device. Some or all elements of the apparatuses can be operatively connected as described in more detail below.

Micro-dialysis catheter 108 can be made of, but is not limited to, flexible, inflexible, or partially flexible material. As discussed above, micro-dialysis catheter 108 can comprise a semipermeable membrane, which will allow selective passage of small molecules (e.g., analyte) from the biological fluid into the inner passageway or lumen of micro-dialysis catheter 108. Micro-dialysis catheter 108 is typically of an appropriate size to allow for rapid equilibration of xenobiotic and/or endogenous compounds or other desired analyte into the dialysis buffer solution that is pumped through micro-dialysis catheter 108.

The portion of micro-dialysis catheter 108 contacting the biological fluid is operatively connected to micro-volume pump 100. As used herein, the term “pump” is a broad term and means, without limitation, a pressurization/pressure device, vacuum device, or any other suitable means for generating fluid flow. In certain embodiments, the pump creates a gradient, thereby facilitating passage of xenobiotic and/or endogenous analytes into the lumen of micro-dialysis catheter 108. In some embodiments, micro-volume pump 100 is a nanoflow pump. The nanoflow pump is a pump optimized for nanoliter-per-minute flows. In other embodiments, micro-volume pump 100 is a microflow pump. The microflow pump is a pump optimized for microliter-per-minute flows. The flow rate of the dialysis buffer solution can range from about 0.1 μL/min to about 500 μL/min depending on the application. It should be appreciated, however, that the scope of the invention is not limited to such flow rate of the dialysis buffer solution. Micro-volume pump 100 can deliver a continuous or a pulsed flow to allow for better equilibration of the dialysis buffer solution and the fluid (e.g., blood) in micro-dialysis catheter 108.

The outlet port of micro-dialysis catheter 108 is optionally connected to second micro-volume pump 112. In certain embodiments, second micro-volume pump 112 is an auto-injector. In some embodiments, second micro-volume pump 112 infuses fluids or compounds into the dialysate or even to the lumen of micro-dialysis catheter 108. Second micro-volume pump 112 can include one or more valves to start, stop, and/or otherwise regulate such delivery. These fluids or compounds can include, but are not limited to, medications, organic solvents, xenobiotics, or other compounds that can be added for quantification or quality control. In one embodiment, second micro-volume pump 112 includes a system for delivering organic solvent. This organic solvent may remove salts or other impurities as part of sample clean-up or preparation (e.g., to improve sample ionization).

Second micro-volume pump 112 can optionally be connected to sample preparation device 116 (e.g., an ionization device for analyzing the sample with a mass spectrometer). When spectrometer device 120 is a mass spectrometer, sample preparation device 116 can be an ionization device. Such a device can include or be coupled to a separation device such as chromatography columns that allows for concentration, clean up or separation of analytes or separation of analytes from unwanted or interfering impurities such as salts. The ionization device converts atoms and/or molecules into ions. In one particular embodiment, the ionization interface is a nano- or micro-flow infusion chip system. This chip system can minimize effects of ion suppression. In various embodiments, the ionization can occur using electrospray, atmospheric pressure chemical ionization, atmospheric photoionization, or other appropriate ionization technologies.

Sample preparation device 116 is operatively connected to spectrometer device 120. Spectrometer device 120 detects the desired analyte, when present in the dialysate, and generates the data that can be analyzed manually or optionally automatically via data analysis device 124. In one particular embodiment, the spectrometry based detection system is a direct infusion tandem mass spectrometer. In other embodiments, the spectrometry based detection system is a quadrupole, orbitrap, time-of-flight, high-field magnets (e.g., Fourier transformation mass spectrometry), sector field mass spectrometry, or other appropriate (mass) spectrometry-based system.

Spectrometry device 120 can optionally be connected to data analysis device 124, such as a computer system. Data analysis device 124 can analyzes the data generated by spectrometer device 120 and present the data in a useful manner, such as a graph or a numeric value. Type of data analysis by data analysis device 124 can include, but are not limited to, generating data on the liberation, metabolism, distribution, absorption, duration, efficacy, toxicity, and/or excretion of a compound. Data analysis device 124 can also include additional data processing such as pattern recognition and database searches. In some embodiments, data analysis device 124 is used to control a feed-back pump system, in which the infusion of a compound into the patient can be regulated.

In use, micro-dialysis catheter 108 is typically inserted into a venous or arterial blood vessel of a patient or other appropriate fluid location. In some embodiments, micro-dialysis catheter 108 is placed intravenously for continuous sampling of analyte and to allow real-time in vivo clinical drug data analysis.

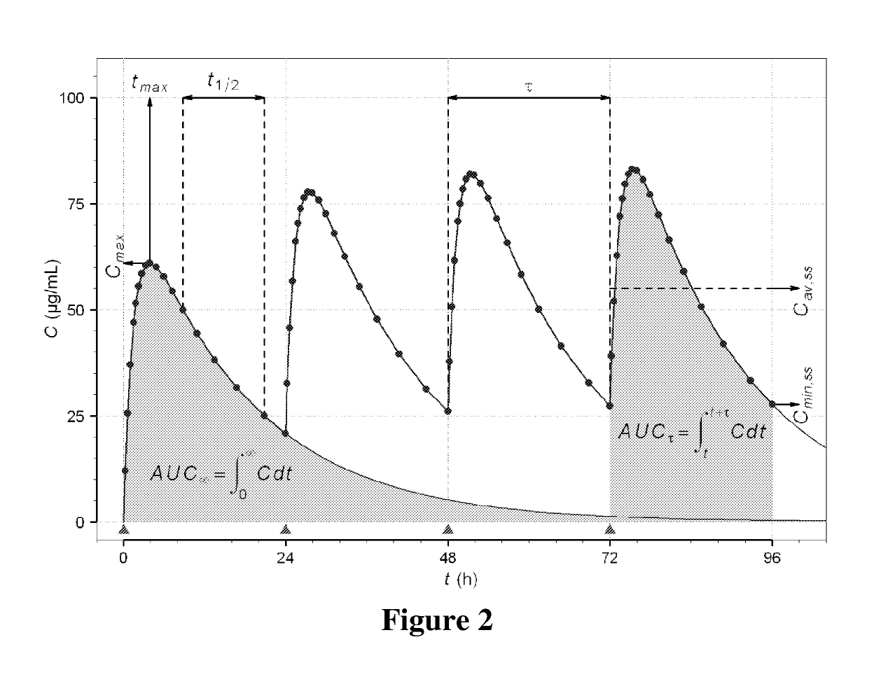

FIG. 2 show a simulated graphic representation of a PK curve that may be obtained by the apparatus or according to a method disclosed herein. The accuracy of these curves and the information they provide such as area under the curve (AUC), half-life (T_{1/2}) and maximum and minimum concentration (Cₘₐₓ, Cₘᵢₙ) are partially or wholly dependent on the accuracy of sampling at critical time points. The accuracy of this data can also influenced by the frequency of sampling. Accordingly, the continuous sampling method disclosed herein provides more accurate in vivo clinical data information.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

This is an in vitro example illustrating the accuracy and usefulness of apparatuses and methods of the invention.

A ten milliliter tube of fresh Na₂EDTA human whole blood was drawn and spiked with 20 μg/mL of acetaminophen. Another ten milliliter tube of control blood was also drawn. A microdialysis (MD) pump (MD 107, CMA) was connected to a microdialysis catheter (MD 64, 64) and to a LC-MS/MS (API5000, AB Sciex) using 0.010″ polyetheretherketone (PEEK) tubing. The MD pump was filled with peritoneal dialysis fluid and the flow rate was set to 5 μL/min. The MD catheter was lowered into the control blood for ten minutes to obtain a baseline reading using a MRM scan.

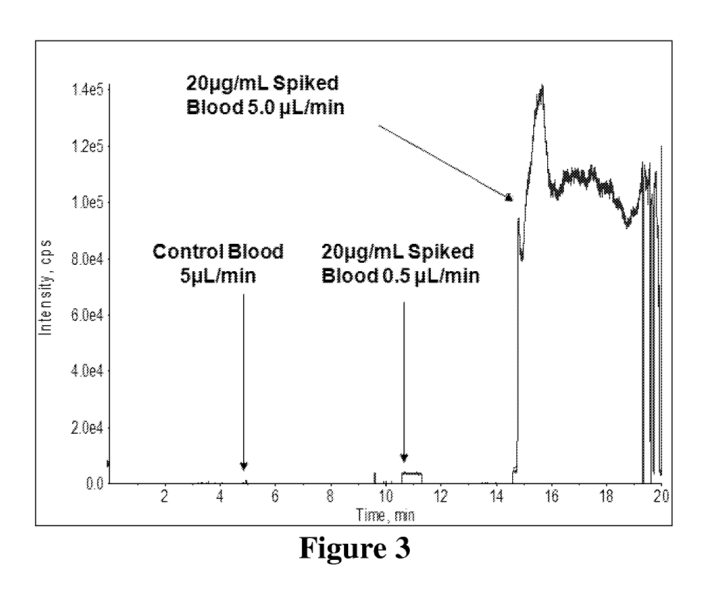

The flow rate was then decreased to 0.5 μL/min and the catheter was switched to the acetaminophen spiked blood. An increase of 10000 cps on the MRM scan was observed (FIG. 3). The catheter was returned to the control blood and the flow rate was set to 5 μL/min. At fourteen minutes the catheter was switched back to the spiked blood with a flow rate 5 μL/min. An increase of 110000 cps on the MRM scan was observed (FIG. 3). The catheter was removed from the spiked blood at 19 minutes.

Methods and apparatuses of the invention provided monitoring of acetaminophen levels in human whole blood using a MD catheter and MD infusion pump flowing at 0.5 μL/min and 5 μL/min with peritoneal dialysis fluid directly introduced into the electrospray source of an LC-MS/MS system in combination with MRM scanning. As shown in FIG. 3, significant increases in MRM signals were observed in the presence of acetaminophen in spiked human whole blood compared to the corresponding MRM signals in acetaminophen-free blood, for example, to 10000 cps for the 0.5 μL/min and 110000 cps for the 5 μL/min flow-rate.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An apparatus comprising:
a micro-volume pump capable of pumping a micro volume of fluid per minute;
a micro-dialysis catheter having an inlet port and an outlet port, wherein said inlet port is operatively connected to said micro-volume pump such that a dialysis buffer solution can be injected or infused into said micro-dialysis catheter through the inlet port using said micro-volume pump to produce a dialysate; and
a spectrometer detection device operatively connected to the outlet port of said micro-dialysis catheter for analyzing the dialysate.

2. The apparatus of claim 1 further comprising an ionization device operatively connected to the outlet port of said micro-dialysis catheter for ionizing at least a portion of the dialysate prior to being analyzed by said spectrometer detection device.

3. The apparatus of claim 1 further comprising a second micro-volume pump operatively connected to the outlet port of said micro-dialysis catheter for transporting the dialysate to said spectrometer detection device.

4. The apparatus of claim 1, wherein said spectrometer detection device comprises a mass spectrometer, a UV/VIS spectrometer, an infrared spectrometer, a chemical, electrochemical or biological sensor, a nuclear magnetic resonance spectrometer or a combination thereof.

5. A method for real-time monitoring of an analyte in a fluid sample of a subject, said method comprising:
injecting or infusing a dialysis buffer fluid using a micro-volume pump through an inlet port of a micro-dialysis catheter that is placed within the subject to produce a dialysate fluid, wherein the micro-dialysis catheter is in fluid communication with the fluid sample of the subject to be analyzed, and wherein the micro-dialysis catheter comprises a barrier medium that allows selective diffusion of the analyte to be detected from the fluid sample of the subject into the lumen of the micro-dialysis catheter and the fluid contained therein; and
analyzing the dialysate fluid using a spectrometer detection device that is operatively connected to an outlet port of the micro-dialysis catheter thereby monitoring the analyte in the fluid sample of the subject.

6. The method for real-time monitoring of an analyte of claim 5, wherein the analyte in the fluid sample is continuously monitored.

7. The method for real-time monitoring of an analyte of claim 5, wherein the analyte in the fluid sample is monitored at least once per hour.

8. The method for real-time monitoring of an analyte of claim 5, wherein the spectrometer detection device comprises a mass spectrometer, a UV/VIS spectrometer, an infrared spectrometer, a chemical, electrochemical or biological sensor, a nuclear magnetic resonance spectrometer or a combination thereof.

9. The method for real-time monitoring of an analyte of claim 8, wherein the spectrometer detection device comprises a mass spectrometer.

10. The method for real-time monitoring of an analyte of claim 9 further comprising flowing at least a portion of the dialysate fluid through an ionization device connected to the outlet port of the micro-dialysis catheter to ionize at least a portion of the dialysate prior to analyzing the dialysate using the mass spectrometer.

11. The method for real-time monitoring of an analyte of claim 10, wherein the ionization device comprises flow infusion chip system.

12. The method for real-time monitoring of an analyte of claim 5 further comprising transporting the dialysate from the outlet port of the micro-dialysis catheter to the spectrometer detection device using a second micro-volume pump operatively connected to the outlet port of the micro-dialysis catheter.

13. The method for real-time monitoring of an analyte of claim 5, wherein the micro-dialysis catheter is placed within a blood vessel of the subject.

14. The method for real-time monitoring of an analyte of claim 13, wherein the micro-dialysis catheter is placed in an arterial or venous blood vessel.

15. The method for real-time monitoring of an analyte of claim 5, wherein the micro-dialysis catheter is placed within the brain, other tissues or the spinal fluid of the subject.

16. The method for real-time monitoring of an analyte of claim 5, wherein the micro-volume pump comprises a nanoflow pump or a microflow pump.

17. A method for obtaining clinical in vivo drug data from a subject comprising:
(a) administering to the subject a drug whose clinical in vivo drug data is to be obtained;
(b) injecting or infusing a dialysis buffer fluid using a micro-volume pump through an inlet port of a micro-dialysis catheter that is placed within the blood vessel of the subject to produce a dialysate fluid, wherein the micro-dialysis catheter comprises a barrier medium that allows selective diffusion of an analyte that is indicative of the drug pharmacokinetics into the lumen of the micro-dialysis catheter and the dialysis fluid contained therein;
(c) analyzing the dialysate fluid using a spectrometer detection device that is operatively connected to an outlet port of the micro-dialysis catheter to determine the level of analyte within the subject's blood; and
(d) repeating steps (b) and (c) to obtain the clinical in vivo drug data from the subject.

18. The method of claim 17, wherein the spectrometer detection device comprises a mass spectrometer, a UV/VIS spectrometer, an infrared spectrometer, a chemical, electrochemical or biological sensor, a nuclear magnetic resonance spectrometer or a combination thereof.

19. The method of claim 18, wherein the spectrometer detection device comprises a mass spectrometer.

20. The method of claim 19 further comprising flowing at least a portion of the dialysate fluid through an ionization device connected to the outlet port of the micro-dialysis catheter to ionize at least a portion of the dialysate prior to analyzing the dialysate using the mass spectrometer.

21. The method of claim 20, wherein the ionization device comprises a flow infusion chip system.

22. The method of claim 17 further comprising transporting the dialysate from the outlet port of the micro-dialysis catheter to the spectrometer detection device using a second micro-volume pump operatively connected to the outlet port of the micro-dialysis catheter.

23. The method of claim 17, wherein the clinical in vivo drug data comprises pharmacokinetics data of the drug.

24. The method of claim 17, wherein the clinical in vivo drug data comprises data associated with the effectiveness of treatment of a clinical condition or toxicity of a drug.
